SEQUENCE LISTING (10 sequences)

SEQ ID NO: 1 - DNA, 18 bp, Artificial Sequence
  Forward primer for VEGF
aactttctgctgtcttgg

SEQ ID NO: 2 - DNA, 18 bp, Artificial Sequence
  Reverse primer for VEGF
tttggtctgcattcacat

SEQ ID NO: 3 - DNA, 20 bp, Artificial Sequence
  Forward primer for aldolase A
gtcatcctcttccatgagac

SEQ ID NO: 4 - DNA, 20 bp, Artificial Sequence
  Reverse primer for aldolase A
aggtagatgtggtggtcact

SEQ ID NO: 5 - DNA, 20 bp, Artificial Sequence
  Forward primer for enolase I
aagaaactgaacgtcacaga

SEQ ID NO: 6 - DNA, 20 bp, Artificial Sequence
  Reverse primer for enolase I
gatcttcgatagacaccact

SEQ ID NO: 7 - DNA, 21 bp, Artificial Sequence
  Forward primer for HIF-1a
ccccagattcaggatcagaca

SEQ ID NO: 8 - DNA, 21 bp, Artificial Sequence
  Reverse primer for HIF-1a
ccatcatgttccatttttcgc

SEQ ID NO: 9 - DNA, 20 bp, Artificial Sequence
  Forward primer for beta-actin
aagagaggcatcctcaccct

SEQ ID NO: 10 - DNA, 20 bp, Artificial Sequence
  Reverse primer for beta-actin
atctcttgctcgaagtccag

(19) United States
(12) Patent Application Publication
(10) Pub. No.: US 2004/0198798 A1
(43) Pub. Date: Oct. 7, 2004

(54) Method for inhibiting tumor angiogenesis and tumor growth

(76) Inventors: Jong-Wan Park, Seoul (KR); Yang-Sook Chun, Seoul (KR); Jinho Kim, Seoul (KR)

Correspondence Address: BURNS, DOANE, SWECKER & MATHIS, L.L.P., P.O. Box 1404, Alexandria, VA, 22313-1404, US

(21) Appl. No.: 10/407,136

(22) Filed: Apr. 7, 2003

(51) Int. Cl. A61K 31/416

(52) U.S. Cl. 514/406

(57) ABSTRACT

The present invention provides methods and pharmaceutical compositions for inhibiting expressions of HIF-1 and HIF-1-regulated genes, angiogenesis, tumor growth, or tumor progression/metastasis comprising contacting the tumor cells or tissue with a composition comprising 3-(5′-hydroxymethyl-2′-furyl)-1-benzylindazole.

### FIELD OF THE INVENTION

The present invention relates to methods and pharmaceutical compositions for inhibiting tumor angiogenesis and tumor growth. We have found that YC-1, 3-(5′-hydroxymethyl-2′-furyl)-1-benzylindazole inhibits hypoxic induction of hypoxia-inducible factor 1α(HIF-1α) transcription factor and the expression of a key angiogenic factor, vascular endothelial growth factor (VEGF) in various human cancer cell-lines in vitro and in vivo. Moreover, YC-1 halts the growth of the xenografted tumors originating from these cancer cell-lines. Therefore, we provide methods and pharmaceutical compositions of using YC-1 for the purpose of treatment of developed tumors and prevention of metastasis and carcinogenesis in animals.

### BACKGROUND OF THE INVENTION

Hypoxia, a reduction in tissue oxygen levels below physiologic levels, commonly develops within solid tumors because tumor cell proliferation is greater than the rate of blood vessel formation. Thus, the increase in tumor mass results in aberrant vasculature formation, which compromises the blood supply (Hockel et al., J Natl Cancer Inst 2001 93:266-276). Tumor hypoxia is one stimulus that leads to the increased expression of vascular endothelial growth factor (VEGF) and stimulates angiogenesis, which is essential for meeting the metabolic requirements of tumor growth (Dachs et al., Eur J Cancer 2000 36:1649-1660). In addition, hypoxia contributes to tumor progression to a more malignant phenotype because cells surviving under hypoxic conditions often become resistant to radiotherapy and chemotherapy (Brown, J. M. Cancer Res 1999 59:5863-5870). Thus, factors that regulate hypoxic events may be good targets for anticancer therapy.

One such target is hypoxia-inducible factor 1 (HIF-1). HIF-1 is a key transcription factor that regulates the blood supply through the expression of vascular endothelial growth factor (VEGF) (Forsythe et al., Mol Cell Biol 1996 16:4604-4613). The biologic activity of HIF-1, a heterodimer composed of HIF-1α and HIF-1β (Wang et al., J Biol Chem 1995 270:1230-1237), depends on the amount of HIF-1α, which is tightly regulated by oxygen tension. Under normoxic conditions, HIF-1α protein is unstable. The instability is regulated, in part, by the binding to the von Hippel-Lindau tumor suppressor protein (pVHL) (Maxwell et al., Nature 1999 399:271-275). This binding occurs after the hydroxylation of the two HIF-1α proline residues by HIF-prolyl hyroxylases (Jaakkola et al., Science 2001 292:468-472; Ivan et al., Science 2001 292:464-468; Masson et al., EMBO J 2001 20:5197-5206). The von Hippel-Lindau protein is one of the components of the multiprotein ubiquitin-E3-ligase complex, which mediates the ubiquitylation of HIF-1α, targeting it for proteasomal proteolysis (Huang et al., Proc Natl Acad Sci U S A 1998 95:7987-7992). However, under hypoxic conditions, proline hydroxylation is inhibited, binding between HIF-1α and the von Hippel-Lindau protein is eliminated and HIF-1α becomes stable.

A growing body of evidence indicates that HIF-1 contributes to tumor progression and metastasis. In human tumors, HIF-1α is overexpressed as a result of intratumoral hypoxia and genetic alterations affecting key oncogenes (HER2, FRAP, HRAS, and CSRC) and tumor suppressor genes (VHL, PTEN, and p53) (Semenza, G. L. Trends Mol Med 2002 8:S62-S67). Immunohistochemical analyses show that HIF-1α is present at higher levels in human tumors than in normal tissues (Zhong et al., Cancer Res 1999 59:5830-5835). Moreover, the expression of HIF-1α in biopsy specimens from various solid tumors has been associated with tumor aggressiveness, vascularity, treatment failure, and mortality (Birner et al., Cancer Res 2000 60:4693-4696). In addition, tumor growth and angiogenesis in xenografted tumors also depends on HIF-1 activity and on the expression level of HIF-1α (Maxwell et al., Proc Natl Acad Sci U S A 1997 94:8104-8109). Therefore, because HIF-1 activity appears central to tumor progression and metastasis, inhibition of HIF-1 activity must be an appropriate anticancer target.

While searching for an antiangiogenic agent that would inhibit HIF-1 activity, we identified a novel pharmacologic action of YC-1. YC-1, 3-(5′-hydroxymethyl-2′-furyl)-1-benzylindazole, inhibits platelet aggregation and vascular contraction by activating soluble guanylyl cyclase, and was originally developed as a potential therapeutic agent for circulation disorders (Teng et al., Eur J Pharmacol 1997 320:161-166; Galle et al., Br J Pharmacol 1999 127:195-203). Recently, we have found a new biological action of YC-1 to block the hypoxic activation of HIF-1 (Chun et al., Biochem Pharmacol 2001 61:947-954). YC-1 reduced HIF-1α expression at the post-transcriptional level and inhibited its transcriptional activity in hepatoma cells cultured under hypoxic conditions. These effects of YC-1 are likely to be linked with the oxygen-sensing pathway, and not with the activation of soluble guanylyl cyclase.

Meanwhile, U.S. Patent Application Publication No. 2002/0040059 A1 discloses methods and formulations for inhibiting and preventing a malignant cell phenotype using a low dose of nitric oxide (NO) mimetic. In detailed description of this US application, it is described that NO mimetic includes any compounds which act as the NO pathway mimetic, however, preferably, the NO mimetic dose not encompass a compound which activates directly either particulate- or soluble guanylyl cyclase, i.e., YC-1, in some embodiments. Practically, the antiangiogenic and antitumorgenic effects of YC-1 have been neither studied nor identified in this US application.

In the present invention, we tested whether YC-1 could target HIF-1 and inhibit tumor angiogenesis in vivo. We confirmed the inhibitory effects of YC-1 on the expression of HIF-1α and on the induction of VEGF, aldolase A, and enolase 1 in cancer cells cultured under hypoxic conditions. In vivo, treatment with YC-1 halted the growth of xenografted tumors originating from hepatoma, stomach carcinoma, renal carcinoma, cervical carcinoma, and neuroblastoma cells. Tumors from YC-1-treated mice showed fewer blood vessels and reduced expression of HIF-1α protein and HIF-1-regulated genes than tumors from vehicle-treated mice. These results support that YC-1 is an inhibitor of HIF-1 that halts tumor growth by blocking tumor angiogenesis and tumor adaptation to hypoxia.

### SUMMARY OF THE INVENTION

The present invention features the antiangiogenic and anticancer effects of YC-1 through the inhibition of HIF-1α.

The present invention provides methods and pharmaceutical compositions for administering YC-1 to animals to inhibit tumor progression and metastasis. Also included in the invention is cancer prevention by administration of YC-1. The invention also provides methods and pharmaceutical compositions for combining YC-1 with other anticancer agents.

The invention further provides methods and pharmaceutical compositions for administering YC-1 to treat a HIF-l-mediated disorder or condition by inactivation of HIF-1.

### BRIEF DESCRIPTION OF THE DRAWINGS

FIG. 1 is an immunoblot showing the effects of YC-1 on the expression of HIF-1α and hypoxia-inducible genes in Hep3B hepatoma cells. Hep3B cells were treated with the indicated concentrations of YC-1 for 5 minutes before being cultured for 4 hours under normoxic (N, 20% )₂ V/V) or hypoxic (H, 1% O₂ V/V) conditions. Expressions of HIF-1α and β-actin proteins were analyzed by immunoblotting with a rabbit anti-HIF-1α antibody and a rabbit anti-β-actin antibody, respectively. Proteins were visualized by enhanced chemiluminescence.

FIG. 2 is an autoradiograph showing mRNA levels of VEGF, aldolase A, enolase 1, HIF-1α, and β-actin. Total RNA was isolated from Hep3B cells that had been treated with the indicated concentrations of YC-1 and cultured under normoxic (N) or hypoxic (H) conditions for 16 hours. mRNA expression was analyzed by semi-quantitative reverse transcription-polymerase chain reaction.

FIG. 3 is a histogram showing the amount of VEGF protein. VEGF protein in conditioned media from Hep3B cells that had been treated with the indicated concentrations of YC-1 and cultured under normoxic (N) or hypoxic (H) conditions for 24 hours was measured using an ELISA kit. The VEGF concentrations were quantified by comparison with a series of VEGF standard samples included in the assay kit. VEGF level in each experiment was measured twice. Bars represent the mean of four separate experiments- with the 95% confidence interval. * denotes statistical significance compared to control supernatants from cells cultured under normoxic conditions (P<0.001); # denotes statistical significance compared to control supernatants from cells cultured under hypoxic conditions (P<0.001).

FIG. 4 is an immunoblot showing the effect of YC-1 on the expression of HIF-1α and VEGF in cancer cells of different origin. NCI-H87 gastric carcinoma, SiHa cervical carcinoma, SK-N-MC neuroblastoma, and Caki-1 renal carcinoma cells were treated with the indicated concentrations of YC-1 for 5 minutes before being cultured under normoxic (N, 20% O₂ V/V) or hypoxic (H, 1% O₂ v/v) conditions for 4 hours. Levels of HIF-1α and β-actin proteins were analyzed by immunoblot analysis using a rabbit anti-HIF-1α antibody or a rabbit anti-β-actin antibody. Proteins were visualized by enhanced chemiluminescence.

FIG. 5 is an autoradiograph showing mRNA levels of VEGF and β-actin in cancer cells of different origin. NCI-H87 gastric carcinoma, SiHa cervical carcinoma, SK-N-MC neuroblastoma, and Caki-1 renal carcinoma cells were treated with the indicated concentrations of YC-1 for 5 minutes before being cultured under normoxic (N, 20% O₂ v/v) or hypoxic (H, 1% O₂ V/V) conditions for 16 hours. mRNA expression was analyzed by semi-quantitative reverse transcription-polymerase chain reaction.

FIG. 6 reveals growth curves of human tumors grafted in the franks of nude mice. Male nude mice were injected subcutaneously in the flank with 5×10⁶ viable Hep3B hepatoma (A), NCI-H87 gastric carcinoma (B), SiHa cervical carcinoma (C), SK-N-MC neuroblastoma (E), or Caki-1 renal carcinoma (F) cells. After the tumors reached 100 to 150 mm³ in size (indicated by long arrows), mice received an intraperitoneal injection of YC-1 (30 μg/g ) or vehicle (DMSO) daily for 2 weeks. Tumor size was measured over time. A) Beginning 2 days after the injection of the Hep3B cells, some mice received injections of YC-1 daily for 2 weeks (indicated by a short arrow). Vehicle=solid circles, YC-1 (established tumors)=open circles, YC-1′ (treatment before established tumors)=open triangles. Each data point represents mean (n=12 for control; n=6 for YC-1; n=7 for YC-1′) and 95% confidence interval. Differences between tumor sizes in the vehicle- and YC-1-treated groups for mice with Hep3B tumors were compared using ANOVA and Duncan's multiple range tests. * denotes P<0.001 relative to the control. Differences between tumor sizes in the vehicle- and YC-1-treated groups for mice with other tumors (B-E) were compared using a Mann-Whitney U test. Each number beneath the error bar represents the P value of the difference relative to the control.

FIG. 7 is a picture showing histopathology for Hep3B hepatoma tumors grown in nude mice. Male nude mice were injected subcutaneously in the flank with 5×10⁶ viable Hep3B cells. After the tumors reached 100 to 150 mm³ in size, mice received an intraperitoneal injection of YC-1 (30 μg/g) or vehicle (DMSO) daily for 2 weeks. After the last treatment, the mice were euthanized, the tumors removed, fixed with formalin, and embedded in paraffin. Tumor sections were cut from the paraffin blocks and stained with hematoxylin and eosin. v, vessel; α, acinus. Scale bar=50 μm.

FIG. 8 is a picture showing vascular distribution in Hep3B hepatoma tumors grown in nude mice. Male nude mice were injected subcutaneously in the flank with 5×10⁶ viable Hep3B cells. After the tumors reached 100 to 150 mm₃ in size, mice received an intraperitoneal injection of YC-1 (30 μg/g) or vehicle (DMSO) daily for 2 weeks. After the last treatment, the mice were euthanized, the tumors removed, fixed with formalin, and embedded in paraffin. Tumor sections were cut from the paraffin blocks and processed for immunohistochemical staining to detect endothelial cells with an anti-CD31 antibody. The immunostained sections were developed using the avidin-biotin-horseradish peroxidase method with diaminobenzidine as the chromagen. The sections were lightly counterstained with hematoxylin. Arrows indicate CD3 1-positive vessels. Scale bar=50 μm.

FIG. 9 is a picture showing expression and distribution of HIF-1α in Hep3B hepatoma tumors grown in nude mice. Male nude mice were injected subcutaneously in the flank with 5×10⁶ viable Hep3B cells. After the tumors reached 100 to 150 mm³ in size, mice received an intraperitoneal injection of YC-1 (30 μg/g) or vehicle (DMSO) daily for 2 weeks. After the last treatment, the mice were euthanized, the tumors removed, fixed with formalin, and embedded in paraffin. Tumor sections were cut from the paraffin blocks and processed for immunohistochemical staining to detect HIF-1α with an anti-HIF-1α antibody. The immunostained sections were developed using the avidin-biotin-horseradish peroxidase method with diaminobenzidine as the chromagen. The sections were lightly counterstained with hematoxylin. Arrows indicate HIF-1α-positive cells. nu, nuclear staining; pn, perinuclear staining. Scale bar=50 μpm.

FIG. 10 is a histogram showing HIF-1α expression detected by immunohistochemistry in human cancer xenografts derived from Hep3B hepatoma, NCI-H87 gastric carcinoma, SiHa cervical carcinoma, SK-N-MC neuroblastoma, or Caki-1 renal carcinoma cells. Male nude mice were injected subcutaneously in the flank with 5×10⁶ viable tumor cells. After the tumors reached 100 to 150 MM³ in size, mice received an intraperitoneal injection of YC-1 (30 μg/g) or vehicle (DMSO) daily for 2 weeks. After the last treatment, the mice were euthanized, the tumors removed, fixed with formalin, embedded in paraffin and processed for immunohistochemistry to detect HIF-1α-positive cells. Two sections per xenograft (5-10 fields per section) were examined for histologic assessment (control and YC-1: n=24 and 12 for Hep3B, n=14 and 12 for NCI-H87, n=10 and 10 for SiHa, n=10 and 10 for SK-N-MC, n=12 and 10 for Caki-1). Each bar represents the mean with lower or upper 95% confidence interval. Differences between treatment groups were compared using a Mann-Whitney U test. Each number over the error bar represents the P value of the difference relative to the control value.

FIG. 11 is a histogram showing vascular density detected by immunohistochemistry in human cancer xenografts derived from Hep3B hepatoma, NCI-H87 gastric carcinoma, SiHa cervical carcinoma, SK-N-MC neuroblastoma, or Caki-1 renal carcinoma cells. Male nude mice were injected subcutaneously in the flank with 5×10⁶ viable tumor cells. After the tumors reached 100 to 150 mm³ in size, mice received an intraperitoneal injection of YC-1 (30 mg/g) or vehicle (DMSO) daily for 2 weeks. After the last treatment, the mice were euthanized, the tumors removed, fixed with formalin, embedded in paraffin and processed for immunohistochemistry to detect CD31-positive cells. Two sections per xenograft (5-10 fields per section) were examined for histologic assessment (control and YC-1: n=24 and 12 for Hep3B, n=14 and 12 for NCI-H87, n=10 and 10 for SiHa, n=10 and 10 for SK-N-MC, n=12 and 10 for Caki-1). Each bar represents the mean with lower or upper 95% confidence interval. Differences between treatment groups were compared using a Mann-Whitney U test. Each number over the error bar represents the P value of the difference relative to the control value.

FIG. 12 is an immunoblot showing the effects of YC-1 on the expression of HIF-1α and VEGF in Hep3B hepatoma cell xenografts. Male nude mice were injected subcutaneously in the flank with 5×10⁶ viable Hep3B cells. After the tumors reached 100 to 150 mm³ in size, mice received an intraperitoneal injection of YC-1 (30 μg/g) or vehicle (DMSO) daily for 2 weeks. After the last treatment, the mice were euthanized, the tumors removed and lysates prepared for immunoblotting. Tumor lysates from vehicle-treated mice (C) and from YC-1-treated (YC-1) mice were assessed by immunoblotting for HIF-1α, VEGF, and and β-actin protein levels.

FIG. 13 is an autoradiograph showing the effects of YC-1 on the expression of hypoxia-inducible genes in Hep3B hepatoma cell xenografts. Male nude mice were injected subcutaneously in the flank with 5×10⁶ viable Hep3B cells. After the tumors reached 100 to 150 mm³ in size, mice received an intraperitoneal injection of YC-1 (30 μg/g) or vehicle (DMSO) daily for 2 weeks. After the last treatment, the mice were euthanized, the tumors removed and lysates prepared for mRNA analysis. The mRNA levels of VEGF, aldolase A, enolase 1, and β-actin were measured by semi-quantitative RT-PCR. The quality of the extracted RNAs was verified by identifying the 18S ribosomal RNA (rRNA) on a 1% denaturing agarose gel.

FIG. 14 is a graph showing the effect of YC-1 on natural killer (NK) cell activity. A) Splenic lymphocytes (6.25×10⁴ to 5×10⁵), isolated from male nude mice, were incubated with YC-1 at various concentrations for 24 hours. The lymphocytes were then incubated at the indicated effector:target cell (E:T) ratios with ⁵¹Cr-labeled YAC-1 cells (1×10⁴). After 4 hours, the amount of radioactivity in the culture supernatants was measured with a gamma counter. B) Male nude mice (n=4 per group) received a daily intraperitoneal injection of vehicle (DMSO) or YC-1 (30 μg/g) for 2 weeks. Splenic lymphocytes were then isolated and tested for NK cell activity. The result is expressed as the mean of four separate experiments with 95% confidence intervals.

### DETAILED DESCRIPTION OF THE INVENTION

The present invention is based on the surprising discovery that YC-1 can substantially inhibit the expressions of HIF-1α and the HIF-1-regulated genes in vitro and in vivo. It is also based on the discovery that YC-1 can have anticancer effect in vivo by blocking tumor angiogenesis essential for tumor growth and metastasis.

Accordingly, one aspect of the present invention provides a method of inhibiting HIF-1α expression in tumor cells or tissues, comprising contacting the tumor cells or tissues with a composition comprising YC-1 at an effective amount for inhibiting HIF-1α.

Another aspect of the present invention provides a method of inhibiting HIF-1-regulated gene expression in tumor cells or tissues, comprising contacting the tumor cells or tissues with a composition comprising YC-1 at an effective amount for inhibiting HIF-1-regulated gene expression.

The other aspect of the present invention provides a method of inhibiting angiogenesis in tumor cells or tissues, comprising contacting the tumor cells or tissues with a composition comprising YC-1 at an effective amount for inhibiting angiogenesis.

A further aspect of the present invention provides a method of inhibiting tumor growth in animal tissues, comprising contacting the animal tissues with a composition comprising YC-1 at an effective amount for inhibiting tumor growth.

Yet another aspect of the present invention provides a method of inhibiting tumor progression and metastasis in animal tissues, comprising contacting the animal tissues with a composition comprising YC-1 at an effective amount for inhibiting tumor progression and metastasis.

A further aspect of the present invention provides a method of treating a HIF-1-mediated disorder or condition in a mammal, comprising administering to the mammal a composition including a therapeutically effective amount of YC-1.

In the present invention, the amount or dosage range of YC-1 employed is one that effectively inhibits expressions of HIF-1α and HIF-1-related genes, angiogenesis, tumor growth, and/or tumor progression and metastasis. It is preferred that the effective amount of YC-1 is 2˜100 μM for inhibiting expressions of HIF-1α and HIF-1-related genes in the cell culture system and 5˜30 μg/g for inhibiting tumor angiogenesis and growth in vivo.

The term “therapeutically effective amount” as used herein indicates an amount of YC-1 that is sufficient to inhibit expressions of HIF-1α0 and HIF-1-regulated genes thereby inhibiting tumor angiogenesis, tumor growth and tumor progression and metastasis without side effects, e.g. apoptosis in cardiac myocyte. As used herein, “HIF-1-related genes” as used herein refer to the genes whose expressions are regulated by HIF-1. The following genes are included in this gene family: erythropoietin, transferrin, transferrin receptor, ceruloplasmin, vascular endothelial growth factor (VEGF), VEGF receptor FLT-1, transforming growth factor β3, plasminogen activator inhibitor 1, α1 B adrenergic receptor, adrenomedullin, endothelin 1, nitric oxide synthase 2, heme oxygenase 1, glucose transporter 1 & 3, hexokinase 1 & 2, enolase 1, glyceraldehyde-3-phosphate dehydrogenase, phosphoglycerate kinase 1, phosphoglucokinase L, pyruvate kinase M, aldolase A & C, trios phosphate isomerase, lactate dehydrogenase A, carbonic anhydrase 9, adenylate kinase 3, prolyl-4-hydroxylase a1, insulin-like growth factor (IGF) 2, IGF-binding protein 1, 2 & 3, P21, Nip3, cyclin G2 and differntiated embryo chondrocyte 1. The term “animal” as used herein is meant to include all mamrnals, and in particular humans. Such animals are also referred to herein as subjects or patients in need of treatment.

The YC-1 according to the present invention may be administered by any suitable route, including orally, parenterally, by inhalation spray, rectally, or topically in dosage unit formulations containing conventional pharmaceutically acceptable carriers, adjuvants, and vehicles. The term “parenteral” as used herein includes, subcutanceous, intravenous, intraarterial, intramuscular, intrasternal, intratendious, intraspinal, intracranial, intrathoracic, infusion techniques or intraperitoneally. In preferred embodiments, the YC-1 is administered intraperitoneally. The dosage administered will be dependent upon the age, health, and weight of the recipient, kind of concurrent treatment, if any, frequency of treatment, and the nature of the effect desired.

The term “pharmaceutically acceptable carrier” as used herein, means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate. Coloring agents, releasing agents, coating agents, sweetening, flavoring and perfuming agents, preservatives and antioxidants can also be present in the pharmaceutical composition of the present invention according to the judgment of the formulator.

The composition comprising YC-1 according to the present invention may be made up in dosage forms such as granules, powders, tablets, pills, capsules, solutions, suspensions, syrups, elixirs, emulsions, ointments, pastes, creams, lotions, gels, sprays, inhalants or patches. The composition of the present invention may be applied in a variety of solutions. Suitable solutions for use in accordance with the present invention are sterile, dissolve sufficient amounts of the YC-1, and are not harmful for the proposed application. Methods of formulation are within the skill of pharmaceutical formulation chemists and are fully described in such works as Remington's Pharmaceutical Science, 18th Edition, Alfonso R. Gennaro, Ed., Mack Publishing Co., Easton, Pa., USA, 1990.

The YC-1, 3-(5′-hydroxymethyl-2′-furyl)-1-benzylindazole, used in the present invention, may be manufactured by prior art techniques or is also available commercially. For example, YC-1 may be obtained from A.G. Scientific Inc. (San Diego, Calif.), Sigma RBI (St Louis, Mo., USA), or Alexis Biochemicals (San Diego, Calif.).

Since the anticancer mechanism of YC-1 quite differs from those of anticancer agents developed previously, the combination of YC-1 with any anticancer agent is expected to be effective. Therefore, YC-1 could be combined with alkylating agents such as asaley, AZQ, BCNU, busulfan, carboxyphthalatoplatinum, CBDCA, CCNU, CHIP, chlorambucil, chlorozotocin, cis-platinum, clomesone, cyanomorpholinodoxorubicin, cyclodisone, dianhydrogalactitol, fluorodopan, hepsulfam, hycanthone, melphalan, methyl CCNU, mitomycin C, mitozolamide, nitrogen mustard, PCNU, piperazine alkylator, piperazinedione, pipobroman, porfiromycin, spirohydantoin mustard, teroxirone, tetraplatin, thio-tepa, triethylenemelamine, uracil nitrogen mustard and Yoshi-864; anitmitotic agents such as allocolchicine, Halichondrin B, colchicines, colchicine derivatives, dolastatin 10, maytansine, rhizoxin, taxol, taxol derivatives, thiocolchicine, trityl cysteine, vinblastine sulfate and vincristine sulfate; topoisomerase I inhibitors such as camptothecin, camptothecin derivatives, aminocamptothecin and morpholinodoxorubicin; topoisomerase II inhibitors such as doxorubicin, amonafide, m-AMSA, anthrapyrazole derivatives, pyrazoloacridine, bisantrene, daunorubicin, deoxydoxorubicin, mitoxantrone, menogaril, N,N-dibenzyl daunomycin, oxanthrazole, rubidazone, VM-26 and VP-16; and antimetabolites such as L-alanosine, 5-azacytidine, 5-fluorouracil, acivicin, aminopterin derivatives, antifol, Baker's soluble antifol, dichlorallyl lawsone, brequinar, ftorafur, 5,6-dihydro-5-azacytidine, methotrexate, methotrexate derivatives, N-(phosphonoacetyl)-L-aspartate (PALA), pyrazofurin, trimetrexate, 3-HP, 5-HP, 2′-deoxy-5-fluorouridine, alpha-TGDR, aphidicolin glycinate, ara-C, 5-aza-2′-deoxycytidine, beta-TGDR, cyclocytidine, guanazole, hydroxyurea, inosine glycodialdehyde, macbecin II, pyrazoloimidazole, thioguanine and thiopurine.

The present invention is broadly applicable to a variety of uses which include inhibition of angiogenesis induced by HIF-1 and treatment of HIF-1-mediated disorders or conditions with accompanying undesired angiogenesis, such as solid and blood-borne tumors including but not limited to melanomas, carcinomas, sarcomas, rhabdomyosarcoma, retinoblastoma., Ewing sarcoma, neuroblastoma, osteosarcoma, and leukemia.

The present invention is more specifically illustrated by the following examples. However, it should be understood that these examples are provided only for illustration of the present invention, but not intended to limit the present invention in any manner.

### BEST MODE FOR CARRYING OUT THE INVENTION

### EXAMPLE 1

### Materials

YC-1, 3-(5′-hydroxymethyl-2′-furyl)-1-benzylindazole, was purchased from A.G. Scientific Inc. (San Diego, Calif.), resuspended in DMSO at a stock concentration of 120 mg/ml, and stored at −30° C. All culture media and fetal bovine serum (FBS) were purchased from Life Technologies (Grand Island, N.Y.).

### EXAMPLE 2

### Cell culture

The Hep3B hepatoma, Caki-1 renal carcinoma, SiHa cervical carcinoma, and SK-N-MC neuroblastoma cell lines were obtained from the American Type Culture Collection (Manassas, Va.). The NCI-H87 stomach carcinoma cell line was obtained from the Korean Cell Line Bank (Seoul, Korea). Hep3B cells were cultured in α-modified Eagle's medium, Caki-1, SiHa, and SK-N-MC cells in Dulbecco's modified Eagle's medium, and NCI-H87 cells in RPMI 1640 medium. All culture media were supplemented with 10% heat-inactivated FBS, 100 units/mL penicillin, and 100 μg/mL streptomycin. All cells were grown in a humidified atmosphere containing 5% CO₂ at 37° C., in which the oxygen tension in the incubator (Vision Sci Co., model 9108MS2, Seoul, KOREA) was held at either 140 mm Hg (20% O₂, v/v, normoxic conditions) or 7 mm Hg (1% O₂, V/V, hypoxic conditions). YAC-1 cell line was obtained from the American Type Culture Collection and maintained in RPMI 1640 medium supplemented with 10% FBS, 1% L-glutamine, 1% nonessential amino acids, 1% sodium pyruvate, 100 units/mL penicillin, and 100 μg/mL streptomycin.

### EXAMPLE 3

### Effect of YC-1 on the expressions of HIF-1α and HIF-1-regulated genes in Hep3B hepatoma cells

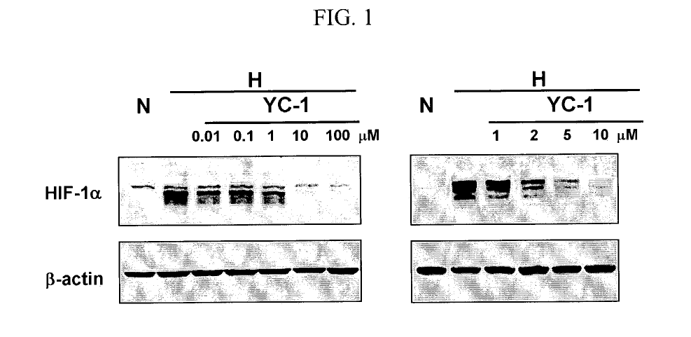

To investigate the inhibitory effect of YC-1 on HIF-1-mediated hypoxic responses, Hep3B cells were treated with YC-1 under hypoxic conditions. Hep3B cells were treated with the indicated concentrations of YC-1 for 5 minutes before being cultured for 4 hours under normoxic (N, 20% O₂ V/V) or hypoxic (H, 1% O₂ V/V) conditions. Immunoblotting was used to detect HIF-1α protein in cultured cells, as described (Chun et al., Biochem Pharmacol 2001 61:947-954). Cells were centrifuged at 3000 rpm for 5 minutes at 4° C. and then washed twice with ice-cold phosphate-buffered saline (PBS). Cells were then resuspended in ten packed cell volumes of a lysis buffer consisting of 10 mM Tris, pH 7.4, 130 mM NaCl, 2 mM EDTA, 1% Nonidet P-40, 0.5 mM dithiothreitol, and 0.5 mM phenylmethylsulfonyl fluoride. Proteins (20 μg) in the cell extract was separated on 6.5% SDS/polyacrylamide gels, and then transferred to an Immobilon-P membrane (Millipore, Bedford, Mass.). Membranes were blocked with 5% nonfat milk in tris-buffered saline containing 0.1% Tween-20 (TTBS) at room temperature for 1 hour and then incubated overnight at 4° C. with rabbit anti-HIF-1α (Chun et al., J Cell Sci 2001 114:4051-4061) diluted 1:1000 in 5% nonfat milk in TTBS. Horseradish peroxidase-conjugated anti-rabbit antiserum (Zymed Laboratories Inc., South San Francisco, Calif.) was used as a secondary antibody (1:5000 dilution in 5% nonfat milk in TTBS, 2 hours incubation) and the antigen-antibody complexes were visualized by using an Enhanced Chemiluminescence Plus kit (Amersham Biosciences Corp., Piscataway, N.J.). As a result, the HIF-1α protein level increased in cells cultured under these conditions for 4 hours without YC-1 but dose-dependently decreased in cells cultured with YC-1 (FIG. 1).

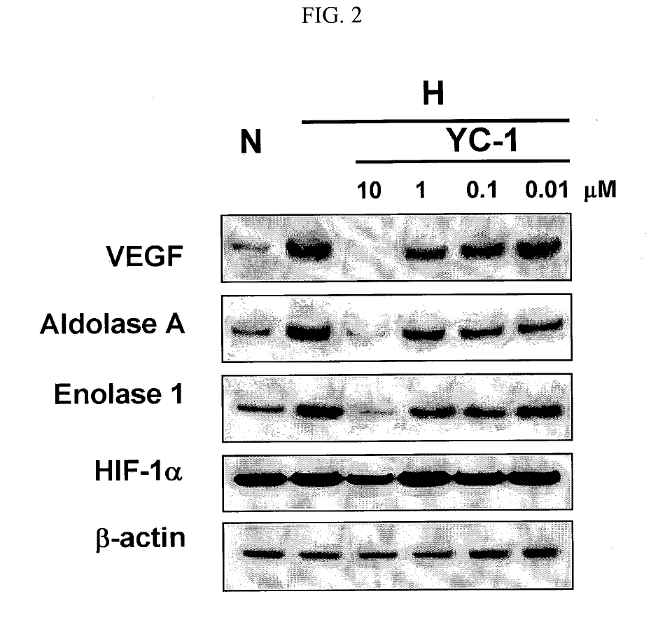

To quantify mRNAs for HIF-1α and HIF-1-regulated genes, we performed a highly sensitive, semi-quantitative reverse transcription-polymerase chain reaction (RT-PCR), as described previously (Chun et al., J Cell Sci 2001 114:4051-4061). Hep3B cells were treated with the indicated concentrations of YC-1 for 5 minutes before being cultured for 16 hours under normoxic (N, 20% O₂ v/v) or hypoxic (H, 1% O₂ v/v) conditions. Total RNAs were isolated from cultured cells using TRIZOL (Life Technologies). After verifying the RNA quality on a 1% denaturing agarose gel, one-μg of total RNA was added to a 50-μL RT-PCR reaction mixture, containing 5 μCi[α(³²P]CTP (NEN Life Science, Boston, Mass.) and 250 nM of each primer pair. The RT-PCR was performed using one cycle of reverse transcription at 48° C. for 1 hour and then 18 PCR cycles, in which one cycle consisted of a denaturation step at 94° C for 30 seconds, an annealing step at 53° C. for 30 seconds, and an elongation step at 68° C. for 1 minute. The resulting PCR fragments (5 μL) were electrophoresed through a 4% polyacrylamide gel at 120 V in a 0.3× Tris-Borate-EDTA (TBE) buffer at 4° C. The gels were dried and then autoradiographed. β-actin mRNA was measured as a PCR control. The nucleotide sequences of the primer pairs (5′ to 3′) were AACTTTCTGCTGTCTTGG (SEQ ID NO: 1) and TTTGGTCTGCATTCACAT (SEQ ID NO: 2) for VEGF, GTCATCCTCTTCCATGAGAC (SEQ ID NO: 3) and AGGTAGATGTGGTGGTCACT (SEQ ID NO: 4) for aldolase A, AAGAAACTGAACGTCACAGA (SEQ ID NO: 5) and GATCTTCGATAGACACCACT (SEQ ID NO: 6) for enolase 1, CCCCAGATTCAGGATCAGACA (SEQ ID NO: 7) and CCATCATGTTCCATTTTTCGC (SEQ ID NO: 8) for HIF-1α, and AAGAGAGGCATCCTCACCCT (SEQ ID NO: 9) and ATCTCTTGCTCGAAGTCCAG (SEQ ID NO: 10) for β-actin. As a result, the expression of HIF-1-regulated genes (VEGF, aldolase A, and enolase 1) dose-dependently decreased in cells cultured with YC-1 for 16 hours, whereas the expression of β-actin mRNA was not affected (FIG. 2). The HIF-1α mRNA level was also relatively unchanged in cells cultured with YC-1, suggesting that YC-1-mediated inhibition of HIF-1α occurs at a post-transcriptional level.

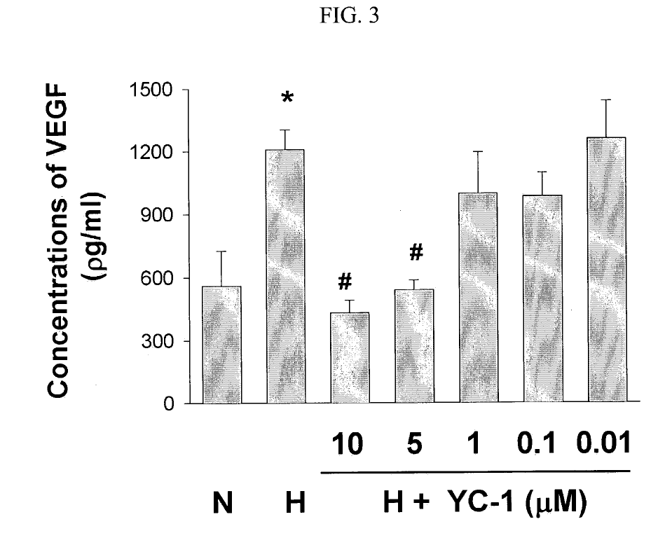

To assess whether the VEGF mRNA levels affected levels of VEGF protein secreted into the medium, we measured VEGF protein levels in Hep3B cell-conditioned medium. For this, Hep3B were plated in a 6-well plate at a density of 1×10⁵ cells/well in α-modified Eagle's medium supplemented with 10% heat-inactivated FBS and incubated overnight. Cells were treated with YC-1 or vehicle (DMSO) for 5 minutes before cells were subjected to normoxia or hypoxia for 24 hours. VEGF levels in the conditioned media were quantified by using the Quantikine human VEGF Immunoassay kit (R&D Systems, Minneapolis, MN) according to the manufacturer's recommended protocol. The VEGF concentrations were quantified by comparison with a series of VEGF standard samples included in the assay kit. After 24 hours, the VEGF protein level in medium from cells cultured under hypoxic conditions (mean=1208 pg/ml, 95% CI=1112 to 1304 pg/ml, <0.001 versus normoxic conditions) was more than twice that from cells cultured under normoxic conditions (mean=559 pg/ml, 95% CI=392 to 726 pg/ml) (FIG. 3). Compared with the VEGF protein level in medium from untreated cells grown under hypoxic conditions, the VEGF protein level in medium from cells cultured with YC-1 decreased in a dose-dependent manner (P<0.001 versus untreated hypoxic conditions) (FIG. 3).

### EXAMPLE 4

### Effect of YC-1 on the expression of HIF-1α and VEGF in cancer cells of different origins

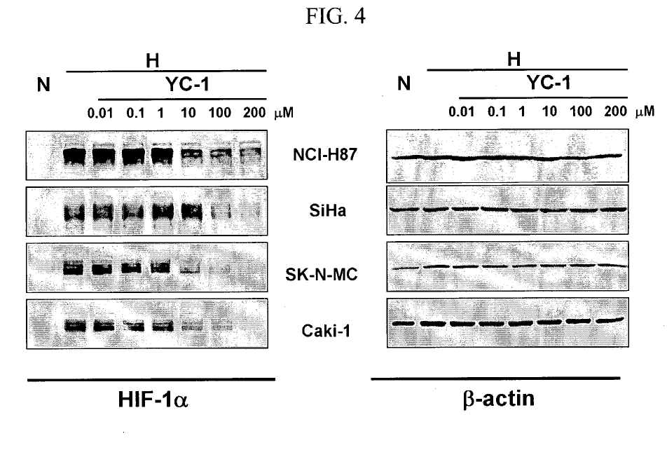

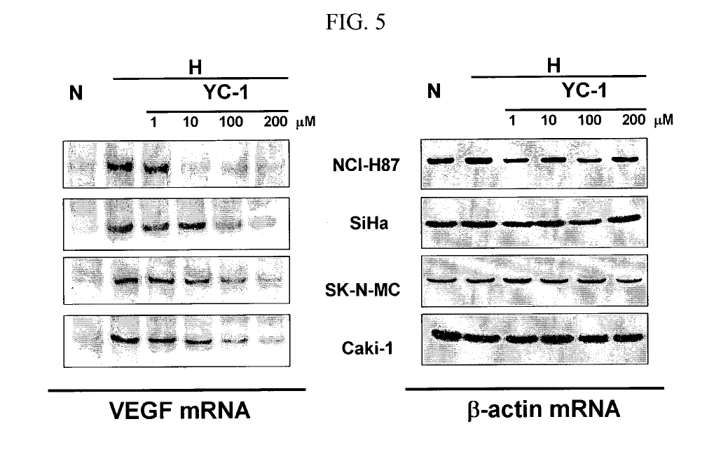

We examined whether the effects of YC-1 were specific to Hep3B cells by assessing the expression of HIF-1α protein and VEGF mRNA in other tumor cell lines (NCI-H87, SiHa, SK-N-MC, and Caki-1) cultured under hypoxic conditions in the absence or presence of YC-1. NCI-H87 gastric carcinoma, SiHa cervical carcinoma, SK-N-MC neuroblastoma, and Caki-1 renal carcinoma cells were treated with the indicated concentrations of YC-1 for 5 minutes before being cultured under normoxic (N, 20% O₂ v/v) or hypoxic (H, 1% O₂ v/v) conditions for 4 hours. Levels of HIF-1α and β-actin proteins were analyzed by immunoblot analysis using a rabbit anti-HIF-1α antibody or a rabbit anti-β-actin antibody. Proteins were visualized by enhanced chemiluminescence. HIF-1α protein and VEGF mRNA were induced in all cell lines cultured under hypoxic conditions in the absence of YC-1 (FIG. 4 and 5). The levels of HIF-1α protein and VEGF mRNA were dose-dependently inhibited in cells cultured under hypoxic conditions in the presence of YC-1. These results confirm that YC-1 inhibits the HIF-1-mediated induction of hypoxia-inducible genes, regardless of the tumor cell type.

### EXAMPLE 5

### Effects of YC-1 on tumor growth in vivo

Because of the observed in vitro effects of YC-1, we investigated whether YC-1 inhibits angiogenesis in solid tumors by suppressing HIF-1, and thus inhibits tumor growth in vivo.

Male nude (BALB/cAnNCrj-nu/nu) mice were purchased from Charles River Japan Inc. (Shin-Yokohama, JAPAN). The animals were housed in a specific pathogen-free room under controlled temperature and humidity. All animal procedures were performed according to the established procedures of the Seoul National University Laboratory Animal Maintenance Manual. Eighty mice aged 7-8 weeks were injected with tumor cells for the xenograft experiments. Sixty nine mice bearing tumors were used for the experiments, but eleven mice were excluded because of technical problems associated with the injection or lack of tumor growth. Twenty five mice were injected subcutaneously in the flank with 5×10⁶ viable Hep3B cells. The mice were immediately randomly assigned to one of three groups. The first group (n=12) was a control group and received the vehicle (DMSO). The second group (n=7) received daily intraperitoneal injections of YC-1 (30 μg/g) beginning the day after the injection of Hep3B cells and continuing for 2 weeks. The third group (n=6) received daily intraperitoneal injections of YC-1 (30 μg/g) for 2 weeks after the Hep3B tumors measured 100-150 mm³. In other experiments, forty four mice were injected with 5×10⁶ NCI-H87, SiHa, SK-N-MC, and Caki-1 tumor cells. Of the mice in each group, 13,10, 10, and 11, respectively, developed tumors. The tumor-bearing mice were randomly assigned to either a control group or an experimental group. After the tumors reached an approximate volume of 100-150 mm³, the mice in the experimental group received daily intraperitoneal injections of YC-1 (30 μg/g) for 2 weeks.

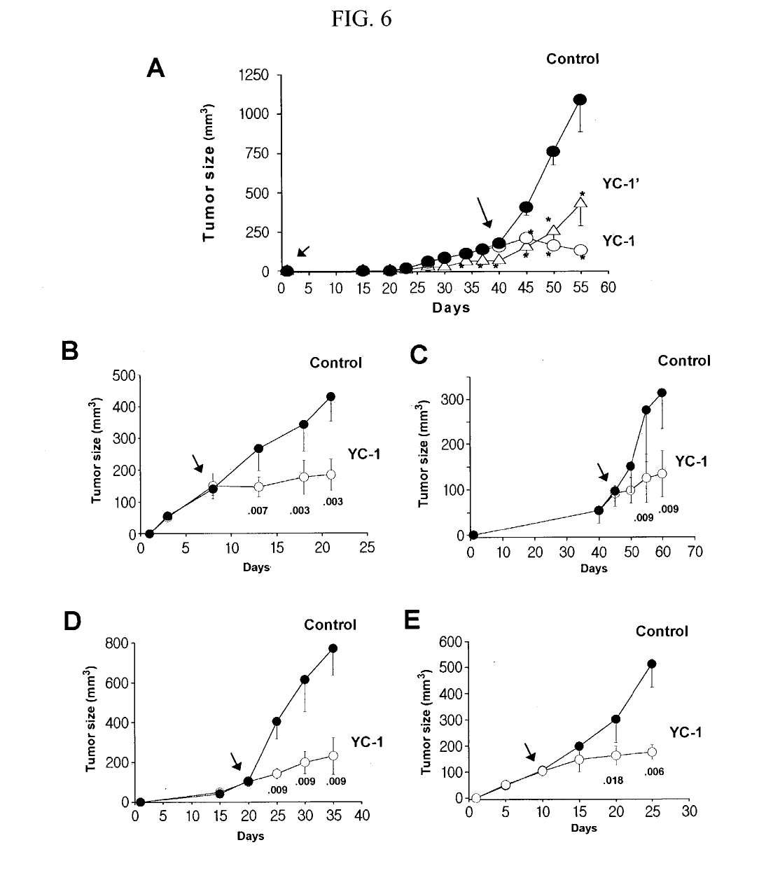

Tumors were measured every 2 or 3 days with calipers in two dimensions and the tumor volumes were calculated using the formula: Volume=a×b²/2, where a is the width at the widest point of the tumor and b is the width perpendicular to a. The results from individual mice were plotted as average tumor volume versus time. As a consequence, tumors in YC-1-treated mice were visibly smaller than those in vehicle-treated mice (FIG. 6). Hep3B tumor growth was minimal in mice treated with YC-1 the day after the tumor cells were injected (the last day of experiment, mean=422 mm³, 95% CI 283 to 561 mm³, P<0.001 versus vehicle-treated group [mean=1082 mm³, 95% CI=880 to 1284 mm³]) and was halted in mice treated with YC-1 after the tumors had become established (mean=126 mm³, 95% CI=97 to 155 mm³, P<0.001 versus vehicle-treated group) (FIG. 6, A). Tumors in mice bearing NCI-H87 (FIG. 6, B), SiHa (FIG. 6, C), SK-N-MC (FIG. 6, D), and Caki-1 (FIG. 6, E) xenografts were also statistically significantly smaller in mice treated with YC-1 than in mice treated with the vehicle (P<0.01 for all comparisons). These results indicate that YC-1 effectively inhibits tumor growth and tumor progression in tumor-bearing mice.

### EXAMPLE 6

### Effects of YC-1 on angiogenesis, HIF-1α protein, and VEGF expression

To determine the mechanism by which YC-1 inhibits tumor growth, we examined Hep3B tumors morphologically and biochemically.

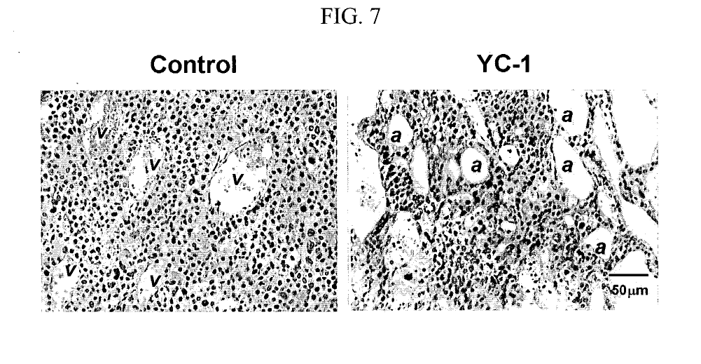

Male nude mice were injected subcutaneously in the flank with 5×10⁶ viable Hep3B cells. After the tumors reached 100 to 150 mm³ in size, mice received an intraperitoneal injection of YC-1 (30 μg/g) or vehicle (DMSO) daily for 2 weeks. After the last treatment, the mice were euthanized, the tumors removed, fixed with formalin, and embedded in paraffin. Serial sections (6-μm thick) were cut from each paraffin block. One section was stained with hematoxylin and eosin (H&E) for histological assessment. Hematoxylin-eosin stained tumor sections from vehicle-treated mice revealed well-developed blood vessels containing red blood cells and frequent mitotic figures (FIG. 7). By contrast, hematoxylin-eosin stained tumor sections from YC-1-treated mice tumors revealed frequent acinus formation without well-developed blood vessels (FIG. 7).

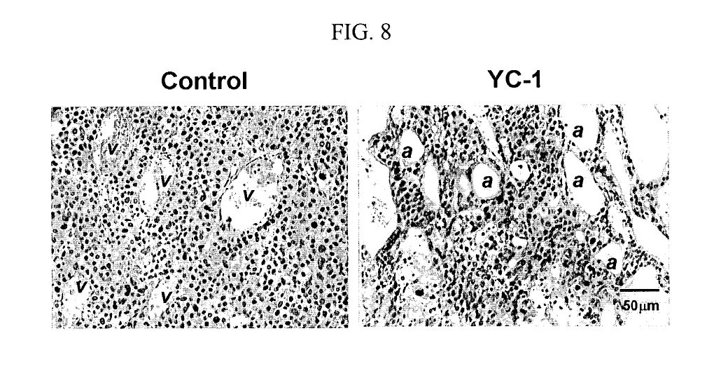

To determine whether the inhibitory effect of YC-1 on tumor growth is associated with the suppression of tumor angiogenesis, we examined the distribution of the endothelial marker, CD31. Other sections were immunochemically stained for HIF-1α and the endothelial cell marker CD31. First, the sections were deparaffinized and rehydrated through a graded alcohol series. Next, the sections were heated in 10 mM sodium citrate (pH 6.0) for 5 minutes in a microwave to retrieve the antigens. After blocking nonspecific sites with a blocking solution containing 2.5% BSA (Sigma-Aldrich Corp., St. Louis, Mo.) and 2% normal goat serum (Life Technologies) in a phosphate-buffered saline (pH 7.4) for 1 hour, the sections were incubated overnight at 4° C. with rabbit polycolnal anti-CD31 (SantaCruz, 1:100 dilution in the blocking solution) or rat anti-HIF-1α (1:100 dilution in the blocking solution) antibodies, as described previously (Kim et al., Circ Res 2002 90:E25-E33). Negative control sections were incubated with diluent in the absence of any primary antibodies. The sections were then stained using standard methods, and the avidin-biotin-horseradish peroxidase complex was used to localize the bound antibodies, with diaminobenzidine as the final chromogen. All immunostained sections were lightly counterstained with hematoxylin. Few CD31-immunopositive vessels were observed in tumor sections from YC-1-treated mice, whereas many vessels were observed in tumor sections from vehicle-treated mice (FIG. 8).

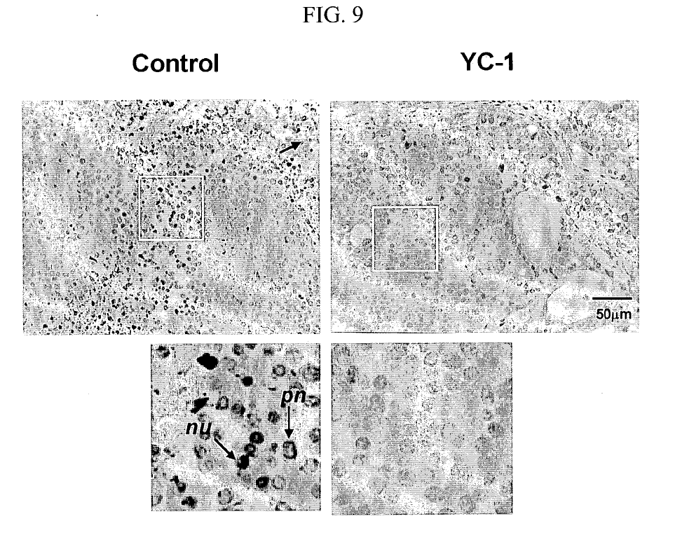

Because HIF-1 is important in angiogenesis, we next assessed HIF-1α expression in tumor sections from vehicle- and YC-1-treated mice (FIG. 9). Hep3B tumors from vehicle-treated mice showed nuclear immunoreactivity (nu) and perinuclear immunoreactivity (pn) for HIF-1α, but only in relatively hypoxic areas away from blood vessels. However, tumor sections from YC-1-treated mice showed no HIF-1α immunoreactive cells (FIG. 9).

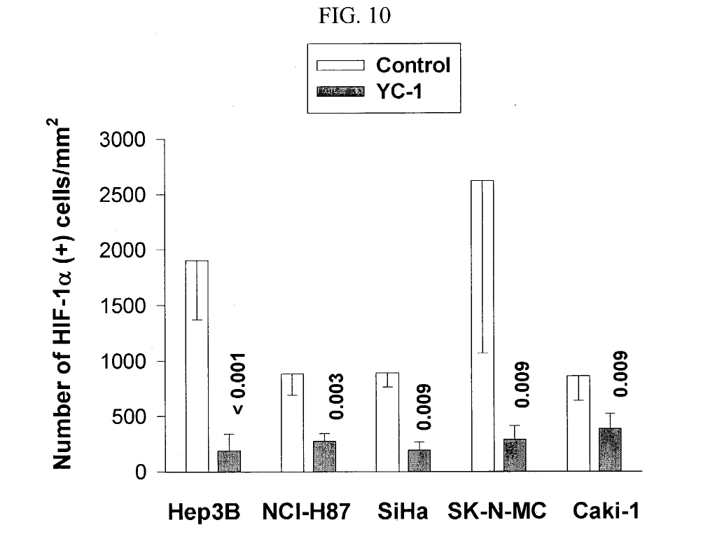

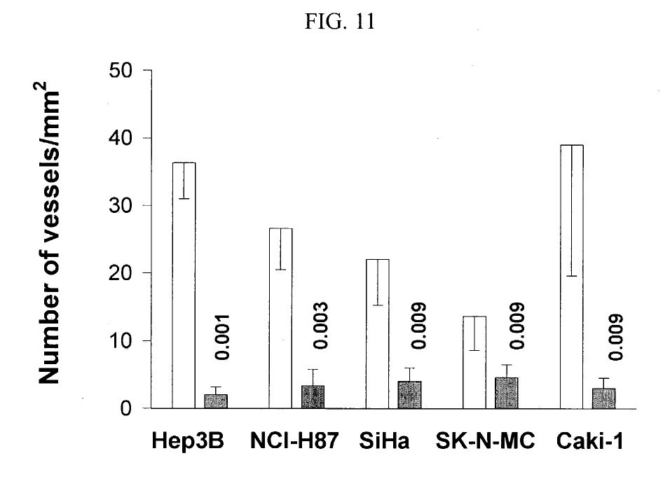

We further quantified the numbers of HIF-1α-positive cells and CD31-positive vessels in tumor sections from vehicle- and YC-1-treated mice. For histological assessment, HIF-1α-positive cells and CD31-positive vessels were identified at magnifications of 200× and 100×, respectively, and examined using a Sony XC-77 CCD camera and a Microcomputer Imaging Device model 4 (MCID-M4) image analysis system. The expression of HIF-1α and the vessel density were measured by counting the numbers of immunopositive cells and vessel profiles (identified by CD31 staining) per mm² under microscopic images. We analyzed ten or more different lesions per xenograft tumor. Regardless of cell origin, the expression of HIF-1α protein and blood vessel formation were statistically significantly inhibited in mice treated with YC-1 for 2 weeks (P<0.01 for all comparisons) (FIG. 10 and 11).

### EXAMPLE 7

### Effect of YC-1 on the Expression of HIF-1α and HIF-1-Regulated Genes in

### Hep3B Hepatoma Cell Xenografts

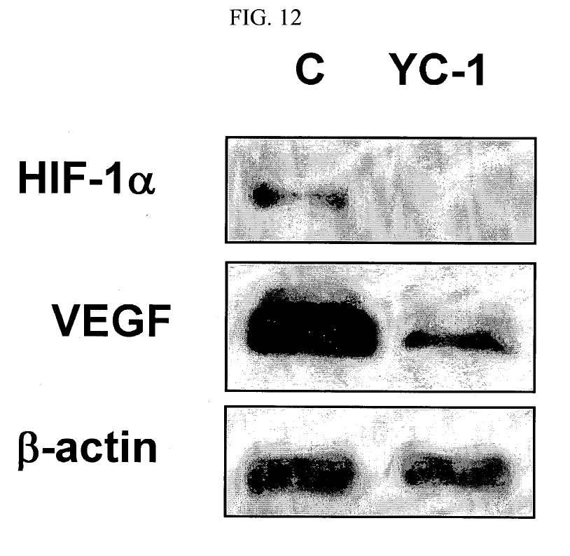

To confirm the effects of YC-1 on the expression HIF-1α and VEGF, we isolated the protein from Hep3B tumor by immunoprecipitation and immunoblotting. Male nude mice were injected subcutaneously in the flank with 5×10⁶ viable Hep3B cells. After the tumors reached 100 to 150 mm³ in size, mice received an intraperitoneal injection of YC-1 (30 μg/g) or vehicle (DMSO) daily for 2 weeks. After the last treatment, the mice were euthanized, the tumors removed and lysates prepared for immunoblotting. Tumor lysates from vehicle-treated mice (C) and from YC-1-treated (YC-1) mice were assessed by immunoblotting for HIF-1α and VEGF protein levels. For the immunoprecipitation of HIF-1α in tumor tissues, tissue lysates in the lysis buffer (150 μg protein) were incubated with 10-μL of the rabbit anti-HIF-1α antiserum overnight at 4° C., and then incubated with protein A-Sepharose beads (Amersham Biosciences Corp.) at a dilution of 1:100 for 2 hours. The antigen-antibody-protein A complexes were washed extensively with the lysis buffer, the immunocomplexes were eluted by boiling for 3 minutes in a sample buffer containing 2% SDS and 10 mM dithiothreitol and subjected to SDS-PAGE, and then immunoblotted using a rat anti-HIF-1α antibody developed previously (Chun et al., Biochem J 2002 362:71-79). β-actin protein was measured as an internal standard. VEGF in tumor tissue was detected using a mouse monoclonal anti-VEGF (SantaCruz Biotecnology Inc., Santa Cruz, Calif.) at a dilution of 1:1000, followed by incubation with a horseradish peroxidase-conjugated anti-mouse antiserum (Zymed Laboratories Inc.). The levels of HIF-1α and VEGF protein expressions were markedly lower in YC-1-treated tumors than in vehicle-treated tumors (FIG. 12).

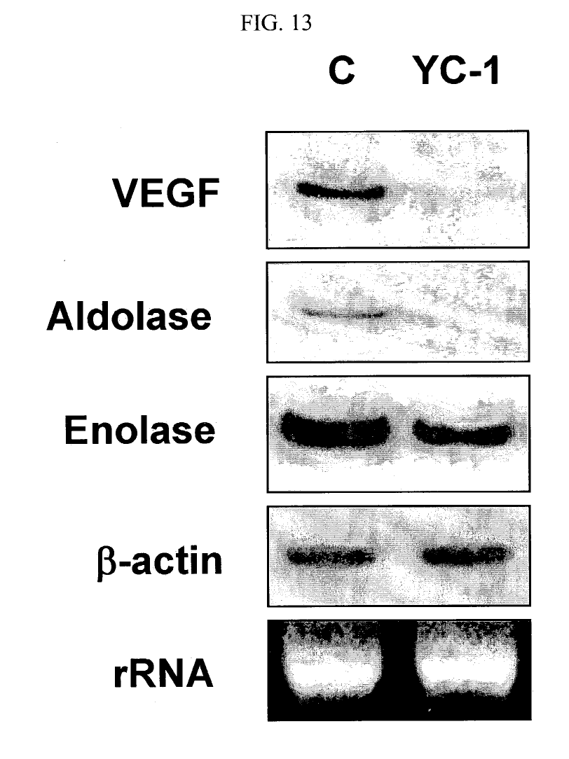

To confirm the effects of YC-1 on the expression of HIF-1-regulated genes in Hep3B hepatoma cell xenografts, we measured the mRNA levels of VEGF, aldolase A, and enolase 1 by semi-quantitative RT-PCR according to the same method as described in EXAMPLE 3. The quality of the extracted RNAs was verified by identifying the 18S ribosomal RNA (rRNA) on a 1% denaturing agarose gel. VEGF, aldolase A and enolase 1 mRNA levels were also lower in YC-1-treated tumors than in vehicle-treated tumors (FIG. 13). The decreased expression of VEGF, aldolase, and enolase may be related to the blocked angiogenesis and the growth retardation observed in YC-1-treated tumors.

### EXAMPLE 8

### Effect of YC-1 on NK-Cell Function

The athymic nude mouse, which has no thymus-dependent immunological functions, is a useful model for assaying tumor growth potential in vivo. However, this mouse has been shown to have thymus-independent NK cells, which are lymphoid cells capable of lysing tumors in the absence of prior stimulation, that have normal cytolytic activity. Thus, to rule out the possibility that YC-1 inhibits tumor growth by activating NK cells, we examined whether YC-1 have an effect on the cytolytic activity of NK cells in vitro and in vivo.

Splenic lymphocytes from 12 nude mice were used to determine the effect of YC-1 on NK cell activity in vitro and in vivo. Individual spleens were homogenized in PBS by passing tissues through a steel mesh using a plunger, and then centrifuged over a Ficoll-Paque (Amersham Biosciences Corp.) gradient at 400×g at room temperature for 30 minutes to isolate the lymphocyte population. The lymphocytes were removed and washed three times in PBS. NK cell activity in the total lymphocyte population was assessed by using a 4-hour ⁵¹Cr-release assay with the NK-sensitive YAC-1 mouse lymphoma cell line as the target cell population, as recently described (Yajima et al., Int J Cancer 2002 99:573-578). YAC-1 cells, grown in RPMI-1640 medium containing 10% FBS, were labeled with sodium chromate (Na_{2 51}CrO₄) at 0.25 mCi/mL for 1.5 hours at 37° C. in a humidified atmosphere containing 5% Co₂.

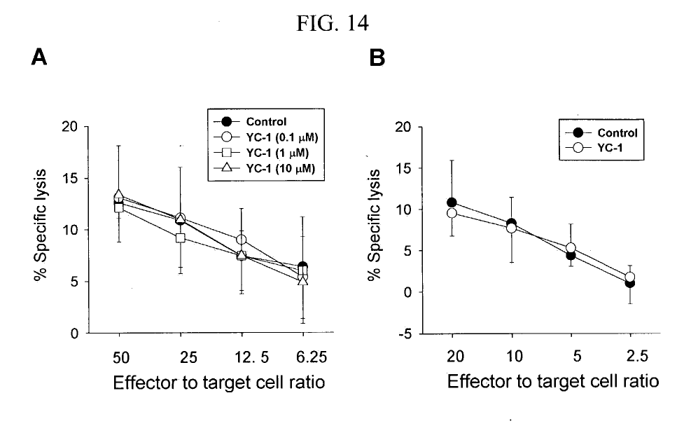

To examine the in vitro effect of YC-1 on NK cell activity, splenic lymphocytes (6.25×10⁴ to 5×10⁵) were incubated with YC-1 (0.1 to 10 μM) or DMSO for 24 hours and then incubated at the indicated effector:target cell (E:T) ratio with 1×10^{4 51}Cr-labeled YAC-1 cells in 96-well round-bottom plates at 37° C. in a humidified atmosphere containing 5% CO₂. After 4 hours, the plates were centrifuged at 200×g at 4° C. for 10 minutes, and 100-μL samples of media were removed and counted for 1 minute in a gamma counter. Splenic lymphocytes taken from a single mouse were used per experiment. Each assay was repeated three times and the average value is the result from one experiment. Results are expressed as the mean and 95% confidence intervals of 4 separate experiments. Splenic lymphocytes incubated with YC-1 in vitro had cytolytic activity against NK-cell sensitive YAC-1 cells that was comparable to that from splenic lymphocytes incubated without YC-1 (FIG. 14, A).

To examine the in vivo effect of YC-1 on NK cell activity, mice received a daily intraperitoneal injection of DMSO (n=4) or of YC-1 (30 μg/g, n=4) for 2 weeks. Splenic lymphocytes were isolated from each mouse and tested immediately for NK cell activity. The spontaneous release of ⁵¹Cr from YAC-1 cells was usually lower than 10% of the total ⁵¹Cr loaded. NK cell activity was calculated as follows: {(experimental release—spontaneous release)/(total release—spontaneous release)}× 100. Each assay was repeated three times and the average value is the result from one experiment. Results are expressed as the mean and 95% confidence intervals of 4 separate experiments. Splenic lymphocytes from mice treated with YC-1 for 2 weeks had cytolytic activity that was comparable to that from vehicle-treated mice (FIG. 14, B).

What is cliamed is:

1. A method of inhibiting HIF-1α expression in tumor cells or tissues, comprising contacting the tumor cells or tissues with a composition comprising 3-(5′-hydroxymethyl-2′-furyl)-1-benzylindazole at an effective amount for inhibiting HIF-1α expression.

2. The method of claim 1, wherein said tumor is selected from the group consisting of hepatoma, stomach carcinoma, renal carcinoma, cervical carcinoma and neuroblastoma.

3. A method of inhibiting HIF-1-regulated gene expression in tumor cells or tissues, comprising contacting the tumor cells or tissues with a composition comprising 3-(5′-hydroxymethyl-2′-furyl)-1-benzylindazole at an effective amount for inhibiting HIF-1-regulated gene expression.

4. The method of claim 3, wherein said HIF-1-regulated gene is selected from the group consisting of erythropoietin, transferrin, transferrin receptor, ceruloplasmin, vascular endothelial growth factor (VEGF), VEGF receptor FLT-1, transforming growth factor β3, plasminogen activator inhibitor 1, α1B adrenergic receptor, adrenomedullin, endothelin 1, nitric oxide synthase 2, heme oxygenase 1, glucose transporter 1 & 3, hexokinase 1 & 2, enolase 1, glyceraldehyde-3-phosphate dehydrogenase, phosphoglycerate kinase 1, phosphoglucokinase L, pyruvate kinase M, aldolase A & C, trios phosphate isomerase, lactate dehydrogenase A, carbonic anhydrase 9, adenylate kinase 3, prolyl-4-hydroxylase al, insulin-like growth factor (IGF) 2, IGF-binding protein 1, 2 & 3, P21, Nip3, cyclin G2 and differntiated embryo chondrocyte 1.

5. The method of claim 4, wherein said HIF-1-regulated gene is selected from the group consisting of VEGF, aldolase A and enolase 1.

6. The method of claim 3, wherein said tumor is selected from the group consisting of hepatoma, stomach carcinoma, renal carcinoma, cervical carcinoma and neuroblastoma.

7. A method of inhibiting angiogenesis in tumor cells or tissues, comprising contacting the tumor cells or tissues with a composition comprising 3-(5′-hydroxymethyl-2′-furyl)-1-benzylindazole at an effective amount for inhibiting angiogenesis.

8. The method of claim 7, wherein said tumor is selected from the group consisting of hepatoma, stomach carcinoma, renal carcinoma, cervical carcinoma and neuroblastoma.

9. A method of inhibiting tumor growth in animal tissues, comprising contacting the tumor cells or tissues with a composition comprising 3-(5′-hydroxymethyl-2′-furyl)-1-benzylindazole at an effective amount for inhibiting tumor growth.

10. The method of claim 9, wherein said tumor is selected from the group consisting of hepatoma, stomach carcinoma, renal carcinoma, cervical carcinoma and neuroblastoma.

11. A method of inhibiting tumor progression and metastasis in animal tissues, comprising contacting the tumor cells or tissues with a composition comprising 3-(5′-hydroxymethyl-2′-furyl)-1-benzylindazole at an effective amount for inhibiting tumor progression and metastasis.

12. The method of claim 11, wherein said tumor is selected from the group consisting of hepatoma, stomach carcinoma, renal carcinoma, cervical carcinoma and neuroblastoma.

13. A method of treating a HIF-1-mediated disorder or condition in a mammal comprising administering to the mammal a composition comprising a therapeutically effective amount of 3-(5′-hydroxymethyl-2′-furyl)-1-benzylindazole.

14. The method of claim 13, wherein said HIF-1-mediated disorder or condition is selected from the group consisting of hepatoma, stomach carcinoma, renal carcinoma, cervical carcinoma and neuroblastoma.

15. A pharmaceutical composition comprising:
a) an amount effective of 3-(5′-hydroxymethyl-2′-furyl)-1-benzylindazole to inhibit tumor angiogenesis, tumor growth or tumor progression and metastasis in a host in need thereof, or to treat an HIF-1-mediated disorder or condition in a mammal; and
b) a pharmaceutically acceptable carrier.
